(19) United States
(12) Patent Application Publication
(10) Pub. No.: US 2009/0143656 A1
(43) Pub. Date: Jun. 4, 2009

(54) SYSTEM FOR DETERMINATION OF BRAIN COMPLIANCE AND ASSOCIATED METHODS

(76) Inventors: Preston Manwaring, Lebanon, NH (US); Kim Manwaring, Phoenix, AZ (US); Mark Manwaring, Payson, UT (US); Jotham Manwaring, Salt Lake City, UT (US)

Correspondence Address: THORPE NORTH & WESTERN, LLP., P.O. Box 1219, SANDY, UT, 84091-1219, US

(21) Appl. No.: 12/064,000

(22) PCT Filed: Aug. 15, 2006
(86) PCT No.: PCT/US06/32020

§ 371 Date: Jul. 31, 2008

Related U.S. Application Data
(60) Provisional application No. 60/708,412, filed on Aug. 15, 2005.

(51) Int. Cl. A61B 5/02; A61B 5/1455; A61B 5/053

(52) U.S. Cl. 600/324; 600/506; 600/504

(57) ABSTRACT

Systems and methods for measuring intracranial pressure and brain compliance are provided. In one aspect, for example, a method for noninvasive measurement of brain compliance in a subject may include calculating a phase shift between an intracranial pulsatile perfusion flow measured from the subject and an extracranial pulsatile perfusion flow measured from the subject, and determining brain compliance of the subject from the phase shift between the intracranial pulsatile perfusion flow and an extracranial pulsatile perfusion flow. Though various methods of calculating phase shift are contemplated, in one aspect such a calculation may include calculating an intracranial frequency waveform corresponding to the intracranial pulsatile perfusion flow, calculating an extracranial frequency waveform corresponding to the extracranial pulsatile perfusion flow, and calculating a phase difference between the intracranial frequency waveform and the extracranial frequency waveform.

### FIELD OF THE INVENTION

The present invention relates to methods and systems for noninvasive determination of brain compliance. Accordingly, this invention involves the fields of neurology, medicine and other health sciences.

### BACKGROUND OF THE INVENTION

The monitoring of intracranial pressure is important in the management of head trauma and many neural disorders. Edema associated with many pathologic conditions of the brain may cause an increase in intracranial pressure that may in turn lead to secondary neurological damage. In addition to head trauma, various neurological disorders may also lead to increased intracranial pressure. Examples of such disorders may include intracerebral hematoma, subarachnoid hemorage, hydrocephalic disorders, infections of the central nervous system, and various lesions to name a few.

As a specific example, congenital hydrocephalus is a disease that causes increased intracranial pressure due to an excess of cerebrospinal fluid, which is often the result of malabsorpition or impediment of clearance in the intraventricular space within the brain or subarachnoid spaces about the brain. If left untreated, hydrocephalus often causes permanent brain damage that may result in deficits of motor skills and learning.

Hydrocephalus is often treated by insertion of a diverting catheter into the ventricles of the brain or into the lumbar cistern. Such a catheter or shunt is connected by a regulating valve to a distal catheter which shunts the spinal fluid to another space where it can be reabsorbed. Examples of common diversion sites include the peritoneum of the abdomen via a ventriculoperitoneal shunt or lumboperitoneal shunt or the atrium of the heart via a ventriculoatrial shunt. Although the symptoms of excessive intracranial pressure and associated ventricular enlargement may be relieved by this procedure, it is not uncommon for the shunt apparatus to become obstructed, resulting in shunt failure. An invasive surgery known as shunt revision may be performed to replace or repair the failed shunt. While shunts may become obstructed at a valve or distal tubing level, a great majority of shunt failures are due to proximal obstruction at the tip of the proximal catheter due to gradual growth of scar about the catheter tip or ingrowth of tissue such as choroid plexus into the catheter tip. A wide variety of techniques of positioning of the catheter and various designs have been explored to diminish obstruction, including many modifications of the side inlet holes of the proximal catheter tip. These have met with modest success at best. The routine clinical approach to shunt failure is therefore to replace the obstructed component and to employ higher pressure regulating valves or related valve components to diminish the tendency of overshunting, a condition characterized by the ventricles eventually becoming much smaller than normal and hugging the proximal catheter.

Regular evaluation of shunt functionality is desirable in the treatment of patients having hydrocephalus. Such functionality may be assessed by measuring brain compliance. One indicator that can be used to evaluate brain compliance is intracranial pressure. As intracranial pressure increases, brain compliance decreases or worsens. It is not always readily apparent to a clinician that a shunt has failed when a patient having a shunt exhibits early shunt failure symptoms such as headache and nausea. Various techniques have been employed to determine functionality of the shunt. For example, an imaging test of the brain such as CT scan, MRI scan, or ultrasound may show progressive ventricular enlargement compared to previous scans. As another example, shunt failure may be demonstrated by inserting a needle into the shunt valve reservoir and attempting to aspirate. An inability to do so may indicate a failed shunt, however a working shunt in very small or slit-like ventricles may act similarly, thus incorrectly reporting that the shunt has failed. As a further example, flow studies such as radioisotope, ultrasound or MRI may show minimal or no flow. Also, a previously implanted intracranial pressure sensor may provide evidence that the shunt has failed or is failing.

The various shunt functionality tests previously utilized may not be preferred in many circumstances due to a high degree of inaccurate results or due to an unnecessary level of invasiveness. In many situations, highly invasive techniques may not be desirable or even possible, as may be the case for many head and other neural traumas where sensors or shunts have not been previously inserted intracranially. Accordingly, systems and methods for accurately determining brain compliance or intracranial pressure would impact the management of hydrocephalus and other neural disorders and head trauma.

### SUMMARY OF THE INVENTION

Accordingly, the present invention provides systems and methods for measuring intracranial pressure. For example, in one aspect a method for noninvasive measurement of brain compliance in a subject is provided. Such a method may include calculating a phase shift between an intracranial pulsatile perfusion flow measured from the subject and an extracranial pulsatile perfusion flow measured from the subject, and determining brain compliance of the subject from the phase shift between the intracranial pulsatile perfusion flow and an extracranial pulsatile perfusion flow. Though various methods of calculating phase shift are contemplated, in one aspect such a calculation may include calculating an intracranial frequency waveform corresponding to the intracranial pulsatile perfusion flow, calculating an extracranial frequency waveform corresponding to the extracranial pulsatile perfusion flow, and calculating a phase difference between the intracranial frequency waveform and the extracranial frequency waveform. In another aspect, at least one of the intracranial frequency waveform or the extracranial frequency waveform may be a sinusoidal frequency waveform.

Numerous methods of measuring pulsatile perfusion flow are contemplated, and thus the present scope should not be limited by those measurement methods exemplified herein. In one aspect, however, at least one of the intracranial pulsatile perfusion flow or the extracranial pulsatile perfusion flow may be measured with an oximeter. In another aspect, at least one of the intracranial pulsatile perfusion flow or the extracranial pulsatile perfusion flow may be measured with an impedance sensor. In yet another aspect, at least one of the intracranial pulsatile perfusion flow or the extracranial pulsatile perfusion flow may be measured with a voltage sensor.

Similarly, various locations for measuring pulsatile perfusion flow are contemplated, and no limitation is intended by those locations exemplified herein. In one aspect, the extracranial pulsatile perfusion flow may be measured from a digital artery in a finger of the subject. In another aspect, the extracranial pulsatile perfusion flow may be measured from an ear of the subject. In yet another aspect, the extracranial pulsatile perfusion flow may be measured from the subject's neck. In a further aspect, the extracranial pulsatile perfusion flow may be obtained from an electrocardiogram of the subject. Regarding measurements of intracranial pulsatile perfusion flow, in one aspect the intracranial pulsatile perfusion flow may be measured from a supraorbital artery of the subject. In another aspect, the intracranial pulsatile perfusion flow may be measured from tympanic membrane displacement. In yet another aspect, the intracranial pulsatile perfusion flow may be measured from retinal tissue of the subject.

It may be beneficial in some cases to provide additional stimuli to facilitate the determination of intracranial pressure. In one aspect, for example, determining brain compliance may further include provoking an increase in intracranial pressure in the subject while measuring intracranial pulsatile perfusion flow and extracranial pulsatile perfusion flow. Though various techniques of accomplishing this are contemplated, in one aspect provoking an increase in intracranial pressure may further includes positioning the subject on a tilt table, tilting the subject to at least two predetermined positions on the tilt table, and calculating a phase difference between the intracranial pulsatile perfusion flow and the extracranial pulsatile perfusion flow for at least one position on the tilt table to determine brain compliance. In another aspect of the present invention, the subject may be tilted to at least three predetermined positions on the tilt table.

The present invention also provides systems for measuring intracranial pressure. For example, in one aspect a system for noninvasive measurement of brain compliance in a subject is provided. Such a system may include a first sensor configured to noninvasively couple to and measure an intracranial pulsatile perfusion flow from the subject, a second sensor configured to noninvasively couple to and measure an extracranial pulsatile perfusion flow from the subject, and a computational device functionally coupled to the first sensor and to the second sensor. The computational device is capable of calculating a phase difference between the intracranial pulsatile perfusion flow and the extracranial pulsatile perfusion flow.

Though numerous sensors are contemplated, in one aspect at least one of the first or the second sensor is an oximeter. In another aspect, at least one of the first or the second sensor is an impedance sensor. In yet another aspect, at least one of the first or the second sensor is a voltage sensor. Additionally, in some aspects the system may further include a display device configured to display the intracranial pulsatile perfusion flow, the extracranial pulsatile perfusion flow, and the phase difference.

Various techniques are contemplated for calculating the phase difference between the intracranial pulsatile perfusion flow and the extracranial pulsatile perfusion flow. Many of such calculations may be facilitated by utilizing sinusoidal representations of the measured waveforms. Accordingly, in one aspect the computation device may be further capable of converting the intracranial pulsatile perfusion flow into an intracranial sinusoidal frequency waveform and the extracranial pulsatile perfusion flow into an extracranial sinusoidal frequency waveform.

Methods of determining abnormal intracranial pressures are also provided by the present invention. In one aspect, for example, a method for noninvasive determination of abnormal intracranial pressure in a subject may include calculating a phase shift between an intracranial pulsatile perfusion flow and an extracranial pulsatile perfusion flow, and comparing the phase shift to a reference phase shift in order to determine abnormal intracranial pressure in the subject. In one specific aspect, the reference phase shift may be a range representing normal intracranial pressures. In one aspect, such a range may be created by calculating a phase shift between an intracranial pulsatile perfusion flow and an extracranial pulsatile perfusion flow from a plurality of humans having intracranial pressure in a normal range. In another aspect, the reference phase shift may be a range representing abnormal intracranial pressures.

### BRIEF DESCRIPTION OF THE DRAWINGS

FIG. 1 is a schematic representation of a system according to an embodiment of the present invention.

FIG. 2 is a graphical representation of data according to another embodiment of the present invention.

FIG. 3 is a graphical representation of data according to yet another embodiment of the present invention.

FIG. 4 is a graphical representation of data according to a further embodiment of the present invention.

### DETAILED DESCRIPTION

### Definitions

In describing and claiming the present invention, the following terminology will be used in accordance with the definitions set forth below.

The singular forms “a,” “an,” and, “the” include plural referents unless the context clearly dictates otherwise. Thus, for example, reference to “a shunt” includes reference to one or more of such shunts, and reference to “an artery” includes reference to one or more of such arteries.

As used herein, “subject” refers to a mammal that may benefit from the administration of a drug composition or method of this invention. Examples of subjects include humans, and may also include other animals such as horses, pigs, cattle, dogs, cats, rabbits, and aquatic mammals.

As used herein, the term “normal” as it relates subjects refers to intracranial pressure or brain compliance levels in a subject that would be determined by one of ordinary skill in the art to not require medical treatment.

As used herein, the term “abnormal” as it relates subjects refers to intracranial pressure or brain compliance levels in a subject that would be determined by one of ordinary skill in the art to require medical treatment, though such medical treatment may not be immediately required.

As used herein, the term “pulsatile perfusion flow” refers to pressure fluctuations of a pulsatile nature that originate from the arterial pulsations of the heart.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

### THE INVENTION

As has been described, various methods of determining intracranial pressure have been utilized in the medical arts, many of which are invasive and/or inaccurate. Accordingly, the present invention provides methods and systems for the noninvasive determination of intracranial pressure, thus also providing a measure of brain compliance. These goals may be accomplished by detecting arterial waveforms from two different arterial flow sensors and mathematically deriving a phase relationship between the two waveforms to yield a curvilinear, positive-correlated value with increased intracranial pressure and worsened compliance.

It has been discovered that intracranial pressure causes a shift in the phase of an intracranial arterial waveform relative to the degree of pressure. Thus by determining the phase shift between an intracranial arterial waveform and an extracranial arterial waveform, intracranial pressure may be accurately determined. Accordingly, in one aspect a method for noninvasive measurement of brain compliance in a subject may include calculating a phase shift between an intracranial pulsatile perfusion flow measured from the subject and an extracranial pulsatile perfusion flow measured from the subject, and determining intracranial pressure or brain compliance of the subject from the phase shift between the intracranial pulsatile perfusion flow and an extracranial pulsatile perfusion flow.

The determination of intracranial pressure from the phase shift between intracranial and extracranial pulsatile perfusion waveforms may be accomplished in various ways. For example, intracranial pressure may be determined by comparing the phase shift obtained from a subject to a reference phase shift or a reference phase shift range. Such a reference may be previously determined from subjects having normal or abnormal intracranial pressure. For example, phase shifts between intracranial and extracranial arterial waveforms may be obtained from a number of subjects known to have normal intracranial pressure levels. These phase shifts can be used to provide a reference against which measured phase shifts can be compared. For example, phase shifts that fall outside of the reference would indicate the likelihood that a subject would have abnormal intracranial pressure. Alternatively, phase shifts between intracranial and extracranial arterial waveforms may be obtained from subjects known to have abnormal intracranial pressure levels. These phase shifts can also be used to provide a reference against which measured phase shifts can be compared. Phase shifts that are similar to this reference would indicate the likelihood that a subject would have abnormal intracranial pressure.

Another method of correlating phase shift to intracranial pressure may utilize a provocative stimulus designed to vary intracranial pressure. In such a method, an increase in intracranial pressure may be provoked while measuring intracranial pulsatile perfusion flow and extracranial pulsatile perfusion flow. By systematically increasing intracranial pressure in an individual, a more accurate determination of how well the individual is regulating intracranial pressure may determined as opposed to a single phase shift value. Numerous methods of increasing intracranial pressure are known. For example, increasing the levels of CO₂ in the blood stream or restricting venous blood flow from the head can cause and increase in intracranial pressure. Such methods may be stressful or painful to many subjects, and thus more comfortable techniques may be preferable.

One example of a more comfortable technique may be to increase intracranial pressure by tilting the head to various positions relative to the heart. As the head is tilted, intracranial blood flow and thus intracranial pressure will be increased or decreased proportional to the relative level of the head to the heart. As the intracranial pressure increases, the phase angle or phase shift is increased between the intracranial arterial waveform and the extracranial arterial waveform. By calculating phase differences between intracranial and extracranial arterial waveforms at various head positions, a plot of phase shifts can be determined for a subject in response to variations in intracranial pressure.

This plot may be compared to reference plots obtained from other subjects. In one aspect, the reference plot may be represented by a number of phase shifts for each tilt position obtained from a number of reference subjects having normal intracranial pressure levels. A statistical range may be determined from the phase shifts of the reference plot in order to provide a comparison with a tested subject. In this case, the plot of phase shift values obtained from the test subject may be compared to the statistical range to determine intracranial pressure abnormalities in the test subject. For example, phase shifts falling outside of the statistical range may be indicative of an intracranial pressure abnormality. In another aspect, a similar statistical range may be determined for individuals having intracranial pressure abnormalities. In these cases, the plot of phase shift values from the test subject is compared against the abnormal statistical ranges to determine intracranial pressure abnormalities. An initial diagnosis of a test subject may be facilitated if the phase shift plot from the test subject falls within a statistical range for a particular abnormality.

Various methods may be utilized to tilt the head to various positions relative to the heart, and all such methods should be considered to be within the scope of the present invention. In one aspect, however, the method may include the use of a tilt table. Such a method may include positioning the subject on the tilt table, tilting the subject to at least two predetermined positions on the tilt table, and calculating a phase difference between the intracranial pulsatile perfusion flow and the extracranial pulsatile perfusion flow for at least one position on the tilt table to determine brain compliance. In another aspect, the subject may be tilted to at least three predetermined positions on the tilt table. In yet another aspect, the subject may be tilted to at least five predetermined positions on the tilt table. In addition to specific predetermined positions, the subject may be tilted continuously from one position to another while measuring phase shift.

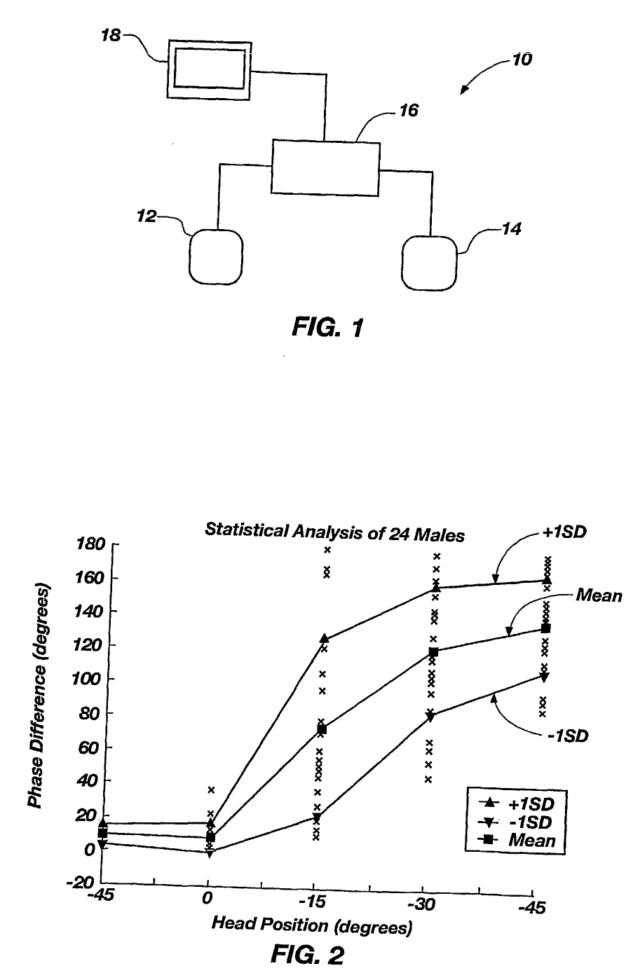

The present invention also provides systems for measuring intracranial pressure and brain compliance. In one aspect, as shown in FIG. 1 for example, a system 10 for noninvasive measurement of brain compliance in a subject is provided. Such a system 10 may include a first sensor 12 configured to noninvasively couple to and measure an intracranial pulsatile perfusion flow from the subject, a second sensor 14 configured to noninvasively couple to and measure an extracranial pulsatile perfusion flow from the subject, and a computational device 16 functionally coupled to the first sensor 12 and to the second sensor 14. The computational device 16 is capable of calculating a phase difference between the intracranial pulsatile perfusion flow and the extracranial pulsatile perfusion flow. Furthermore, such a system 10 may further include a display device 18 configured to display the intracranial pulsatile perfusion flow, the extracranial pulsatile perfusion flow, and the calculated phase shift or phase difference between the intracranial pulsatile perfusion flow and the extracranial pulsatile perfusion flow. In one aspect, the computation device 16 may further be capable of converting the intracranial pulsatile perfusion flow into an intracranial sinusoidal frequency waveform and the extracranial pulsatile perfusion flow into an extracranial sinusoidal frequency waveform. Such a conversion may facilitate the calculation of the phase difference, which is described below.

Various measurement locations for the intracranial pulsatile perfusion flow are contemplated. It should be noted, however, that any measurement location where intracranial pulsatile perfusion flow can be determined noninvasively would be considered to be within the scope of the present invention. In some aspects, for example, the intracranial pulsatile perfusion flow can be measured from a location that is outside of the cranium. In one aspect intracranial pulsatile perfusion flow may be measured from the supraorbital artery, derived from the internal carotid artery. The intracranial internal carotid artery bifurcates into two branches, one of which is the ophthalmic artery. This artery exits the intracranial space to become the supraorbital artery, which passes over the forehead through the supraorbital foramen and above the ocular globe. Intracranial arterial pulsations are altered by intracranial pressure and brain compliance or stiffness, and such alterations in waveform are manifest downstream in the course of the supraorbital artery where it exits into the plane beneath the scalp. Thus the supraorbital artery may provide a measurement of intracranial pulsatile perfusion flow through measurement at the forehead of the subject.

In another aspect, intracranial pulsatile perfusion flow may be measured by detection of tympanic membrane displacement, a pulsatile pattern corresponding to the intracranial pulsatile perfusion flow. Such measurement may occur by, for example, placing a tympanic membrane displacement sensor into the external ear canal of one ear of the subject. Intracranial pulsation is thus transmitted through the middle ear bones to the tympanum, and thus to the sensor located in the external ear canal.

Other methods of measuring intracranial pulsatile perfusion flow may also be utilized, such as, without limitation, measurements from retinal tissue, measurements from MRI or other neural imaging devices, ultrasound, etc.

Various devices are contemplated for the noninvasive measurement of intracranial pulsatile perfusion flow. It should be noted that any device capable of measuring such an intracranial pulsatile perfusion flow should be considered to be within the scope of the present invention. Examples may include, without limitation, oximeters, impedance sensors, voltage sensors, transcranial current impedance sensors, infrared transmission or reflectance sensors, and combinations thereof. In one specific aspect, an oximeter may be fixed to the forehead of a subject in order to measure intracranial pulsatile perfusion flow from the supraorbital artery of the subject.

Numerous techniques for measuring extracranial pulsatile perfusion flow are contemplated, all of which are considered to be within the scope of the present invention. Extracranial pulsatile perfusion flow measurements may be obtained from virtually any arterial location originating from outside of the cranium. Such measurement locations and techniques are very well known to those of ordinary skill in the art, and as such, they will not be discussed in great detail. Various examples may include, without limitation, arteries of the fingers, hands, arms, legs, and feet, arteries of the neck and head such as external carotid arteries, electrocardiograms (ECGs), and combinations thereof. Specific examples may include arteries of the fingers, arteries of the earlobes, arteries of the neck, and combinations thereof.

Various devices are also contemplated for the noninvasive measurement of extracranial pulsatile perfusion flow. It should be noted that any device capable of measuring such an intracranial pulsatile perfusion flow should be considered to be within the scope of the present invention. Examples may include, without limitation, oximeters, impedance sensors, voltage sensors, transcranial current impedance sensors, infrared transmission or reflectance sensors, and combinations thereof. In one specific aspect, an oximeter may be fixed to the finger of a subject in order to measure extracranial pulsatile perfusion flow from a digital artery of the subject. In another specific aspect, an oximeter may be fixed to the ear of a subject in order to measure extracranial pulsatile perfusion flow from the subject. In yet another aspect, a voltage sensor may be utilized to measure an ECG waveform from the subject, from which the extracranial pulsatile perfusion flow may be obtained.

The comparison of intracranial arterial flow and extracranial arterial flow may provide an accurate measurement of intracranial pressure and brain compliance in a subject. As has been described, a first sensor measures an intracranial arterial waveform that has been affected by intracranial pressure and brain compliance. A second sensor measures an extracranial arterial waveform that has not been affected by intracranial pressure or brain compliance. One result of the affects of the intracranial pressure on the intracranial arterial waveform is a phase shifting relative to the extracranial waveform to a degree that is proportional to the level of intracranial pressure.

Any method of calculating the phase shift between the waveforms is to be considered within the present scope. In one aspect, however, calculating phase shift may include calculating an intracranial frequency waveform corresponding to the intracranial pulsatile perfusion flow, calculating an extracranial frequency waveform corresponding to the extracranial pulsatile perfusion flow, and calculating a phase difference between the intracranial frequency waveform and the extracranial frequency waveform. In one aspect, the frequency waveform may be a sinusoidal frequency waveform. Such waveforms may be conveniently obtained from a fast Fourier transformation (FFT) function. FFTs are commonly used algorithms for converting time domain sampled data into frequency domain data. The frequency domain data from both sampled waveforms is useful for identifying the characteristics which determine brain compliance. FFTs are well know in the art, and any such algorithm may be utilized to obtain sinusoidal frequencies for which phase shifts may be obtained. One such algorithm is discussed in the Examples below.

### EXAMPLES

The following examples are provided to promote a more clear understanding of certain embodiments of the present invention, and are in no way meant as a limitation thereon.

### Example 1

A subject was positioned on a tilt table having a motorized solenoid mechanism that moves the table in order to invert the subject in sequential steps or continuous movement from about +45° head up to a −45° head down position. A pulse-oximeter (MAXFAST® Nellcor, Pleasanton Calif.) was attached to the forehead of the subject over a supraorbital artery. A clip-type pulse-oximeter (Nellcor, Pleasanton Calif.) was attached to a finger of the subject to record from a digital artery. The arm of the subject to which the finger oximeter is attached is placed over the subject's heart to minimize phase shifts due to pressure changes in the arterial system. Voltage outputs from each oximeter are connected to a data acquisition system (DAQCard-6036E, National Instruments, Austin, Tex., USA). The subject was sequentially tilted to specific positions by movement of the table. These positions were +45°, 0°, −15°, −30°, and −45°. The subject was held for 30 seconds at each position to stabilize heart rate. After stabilization, 30 seconds of data were recorded. After recording, the subject was advanced to the next position. Intracranial arterial waveforms and extracranial arterial waveforms were recorded at each of the positions indicated.

### Example 2

A subject was prepared as indicated in Example 1. The subject was slowly advanced continuously from +45° to −45° and back to +45° over a period of 2 minutes. Data was collected for the full 2 minute duration. Data processing was carried out with a continuously moving FFT window of 3 seconds over the experiment duration. Data points before time zero were zero padded for the FFT.

### Example 3

The following FFT algorithm was utilized to calculate phase shifts between waveforms obtained in Example 1. First, the heart rate of the subject was determined by finding the frequency bin with the maximum value. This frequency is the same for both the intracranial waveform and the extracranial waveform. Second, the average phase angle over the sample period of each waveform was calculated from the complex value of the bins of the previous step. Finally, the phase angles of the two waveforms were subtracted from each other. This phase difference changes with increasing intracranial pressure and is a measure of brain compliance.

This algorithm was used in an automated data acquisition and analysis software package developed in MATLAB (MathWorks, Natick, Mass., USA) and customized for this application. The scripts first simultaneously acquire 30 seconds of 16-bit 100 samples/second data from each sensor. Once complete, the data is bandpass filtered and analyzed using a 1024 point FFT. Assuming clean waveforms, the peak values (∥Real+Imaginary∥) found in the FFT bin sets will be the fundamental (sinusoidal decomposition) frequency of pulsatility in the brain. The phase of a single sinusoid is then given as

Phase=tan⁻¹ Imaginary/Real

The phase relationship between the fundamental frequencies (assuming they are the same frequency) is then given as

PhaseDifference=Phase₁−Phase₂

MATLAB scripts were written to guide technicians through the data-acquisition process with participants. Processing of this data was as described above and took place after data acquisition was completed.

### Example 4

A group of 24 male subjects having no history of hydrocephalus were evaluated for intracranial pressure. Each subject was evaluated as described in Example 1. FIG. 2 shows the phase angles of the 5 sequential positions tested for each subject. FIG. 2 further shows a +1 standard deviation (+1 SD) line and a −1 standard deviation (−1 SD) line and a mean line plotted for reference. The range between the +1 SD and the −1 SD represent phase shifts indicating normal intracranial pressure levels in a subject.

### Example 5

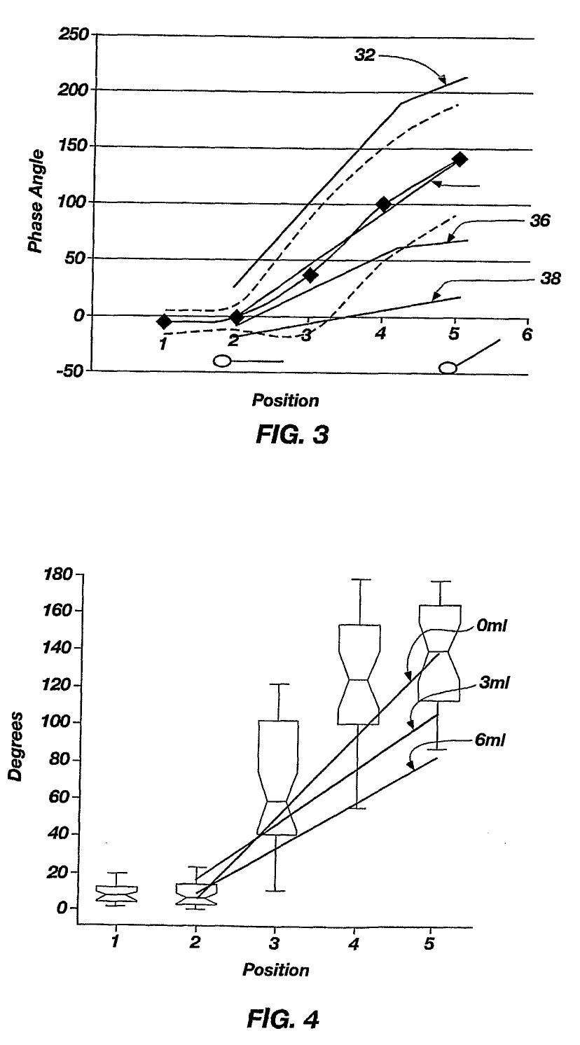

A group of twenty subjects with hydrocephalus in various stages of diagnosis and treatment were evaluated as in Example 1. FIG. 3 shows four patterns of phase shift compared to normal subjects as cited in Example 4. Patients may be classified as having abnormally low compliance with a plot consistent with a failed or obstructed shunt and thus above +1 SD compared to normal subjects 32; within normal range with acceptably functional shunts 34; falling out of normal range on tilting to excessively low compliance, suggesting a need for modification of the shunt valve 36; and outside of normal pressure range with excessively high compliance such as may be seen in an overdraining shunt or in normal pressure hydrocephalus 38.

### Example 6

A subject with hydrocephalus had an implanted Ommaya ventricular tapping reservoir that had been accessed with percutaneous puncture with a needle and a manometer apparatus to allow monitoring of intracranial pressure. The subject was sequentially evaluated at the 5 positions as in Example 1. Following the withdrawal of 3 ml of cerebrospinal fluid (CSF), the subject was again evaluated at the 5 positions as in Example 1. 3 ml of CSF was again withdrawn and the evaluation of Example 1 were again repeated. FIG. 4 shows the phase shift for each tilt table position of the subject for 0 ml, 3 ml, and 6 ml of CSF withdrawn.

It is to be understood that the above-described systems and methods are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A method for noninvasive measurement of brain compliance in a subject, comprising:
calculating a phase shift between an intracranial pulsatile perfusion flow measured from the subject and an extracranial pulsatile perfusion flow measured from the subject; and
determining brain compliance of the subject from the phase shift between the intracranial pulsatile perfusion flow and an extracranial pulsatile perfusion flow.

2. The method of claim 1, wherein calculating a phase shift further includes:
calculating an intracranial frequency waveform corresponding to the intracranial pulsatile perfusion flow;
calculating an extracranial frequency waveform corresponding to the extracranial pulsatile perfusion flow; and
calculating a phase difference between the intracranial frequency waveform and the extracranial frequency waveform.

3. The method of claim 1, wherein at least one of the intracranial frequency waveform or the extracranial frequency waveform is a sinusoidal frequency waveform.

4. The method of claim 1, wherein at least one of the intracranial pulsatile perfusion flow or the extracranial pulsatile perfusion flow is measured with an oximeter.

5. The method of claim 1, wherein at least one of the intracranial pulsatile perfusion flow or the extracranial pulsatile perfusion flow is measured with an impedance sensor.

6. The method of claim 1, wherein at least one of the intracranial pulsatile perfusion flow or the extracranial pulsatile perfusion flow is measured with a voltage sensor.

7. The method of claim 1, wherein the extracranial pulsatile perfusion flow is measured from a digital artery in a finger of the subject.

8. The method of claim 1, wherein the extracranial pulsatile perfusion flow is measured from an ear of the subject.

9. The method of claim 1, wherein the extracranial pulsatile perfusion flow is measured from the subject's neck.

10. The method of claim 1, wherein the extracranial pulsatile perfusion flow is obtained from an electrocardiogram of the subject.

11. The method of claim 1, wherein the intracranial pulsatile perfusion flow is measured from a supraorbital artery of the subject.

12. The method of claim 1, wherein the intracranial pulsatile perfusion flow is measured from tympanic membrane displacement.

13. The method of claim 1, wherein the intracranial pulsatile perfusion flow is measured from retinal tissue of the subject.

14. The method of claim 1, wherein determining brain compliance further includes provoking an increase in intracranial pressure while measuring intracranial pulsatile perfusion flow and extracranial pulsatile perfusion flow.

15. The method of claim 14, wherein provoking an increase in intracranial pressure further includes:
positioning the subject on a tilt table;
tilting the subject to at least two predetermined positions on the tilt table;
calculating a phase difference between the intracranial pulsatile perfusion flow and the extracranial pulsatile perfusion flow for at least one position on the tilt table to determine brain compliance.

16. The method of claim 15, wherein the subject is tilted to at least three predetermined positions on the tilt table.

17. The method of claim 1, further comprising:
displaying the intracranial pulsatile perfusion flow and the extracranial pulsatile perfusion flow; and
displaying the phase difference between the intracranial pulsatile perfusion flow and the extracranial pulsatile perfusion flow.

18. A system for noninvasive measurement of brain compliance in a subject, comprising:
a first sensor configured to noninvasively couple to and measure an intracranial pulsatile perfusion flow from the subject;
a second sensor configured to noninvasively couple to and measure an extracranial pulsatile perfusion flow from the subject; and
a computational device functionally coupled to the first sensor and to the second sensor, said computational device being capable of calculating a phase difference between the intracranial pulsatile perfusion flow and the extracranial pulsatile perfusion flow.

19. The system of claim 18, wherein at least one of the first or the second sensor is an oximeter.

20. The system of claim 18, wherein at least one of the first or the second sensor is an impedance sensor.

21. The system of claim 18, wherein at least one of the first or the second sensor is a voltage sensor.

22. The system of claim 18, further comprising a display device configured to display the intracranial pulsatile perfusion flow, the extracranial pulsatile perfusion flow, and the phase difference.

23. The system of claim 18, wherein the computation device is further capable of converting the intracranial pulsatile perfusion flow into an intracranial sinusoidal frequency waveform and the extracranial pulsatile perfusion flow into an extracranial sinusoidal frequency waveform.

24. A method for noninvasive determination of abnormal intracranial pressure in a subject, comprising:
calculating a phase shift between an intracranial pulsatile perfusion flow and an extracranial pulsatile perfusion flow; and
comparing the phase shift to a reference phase shift in order to determine abnormal intracranial pressure in the subject.

25. The method of claim 24, wherein the reference phase shift is a range representing normal intracranial pressures.

26. The method of claim 25, wherein the range representing normal intracranial pressures is created by calculating a phase shift between an intracranial pulsatile perfusion flow and an extracranial pulsatile perfusion flow from a plurality of humans having intracranial pressure in a normal range.

27. The method of claim 24, wherein the reference phase shift is a range representing abnormal intracranial pressures.
